(19) United States
(12) Patent Application Publication
(10) Pub. No.: US 2022/0047217 A1
(43) Pub. Date: Feb. 17, 2022

(54) SKIN PATCH

(71) Applicant: SM24 LIMITED, London (GB)

(72) Inventors: James Mayo, Scottsdale, AZ (US); Martyn Boutelle, London (GB)

(21) Appl. No.: 17/278,955

(22) PCT Filed: Sep. 27, 2019
(86) PCT No.: PCT/GB2019/052735

§ 371 Date: Mar. 23, 2021

(30) Foreign Application Priority Data
Sep. 27, 2018 (GB) 1815736.2
Dec. 7, 2018 (GB) 1819998.4

(51) Int. Cl. A61B 5/00; A61B 5/145

(52) U.S. Cl. CPC A61B 5/6833; A61B 5/14517; A61B 2560/0223; A61B 5/14532; A61B 5/002

(57) ABSTRACT

The present invention provides a skin patch for measuring a biometric parameter of a bodily fluid. The skin patch comprises a hydrophilic sensing bead and a sensor including at least one electrode embedded within the hydrophilic sensing bead, in which the sensor is configured to obtain data relating to the concentration of the hydrophilic sensing bead relating to the parameter. The skin patch further comprises a transport section for transporting a bodily fluid to contact the hydrophilic sensing bead. The skin patch also comprises a communication unit, which includes an input section communicating with the sensor via a connector, and an output section configured to output the obtained data.

### FIELD OF THE INVENTION

The present invention relates to a skin patch for measuring a biometric parameter of a bodily fluid, and a kit thereof.

### BACKGROUND

With fitness regimes becoming ever more popular, there has been a lot of focus on personalising training. Sweat analysis in particular gives an indication of health and fitness. Much of the prior art aims to collect sweat and then subsequently analyse the collected sweat in a laboratory environment. However, the average sweat rate amounts only to approximately 5 nl per minute per gland. With only 100 glands per cm², this poses the problem that it is difficult to collect a sufficient amount of sweat for laboratory analysis and that sweat is not fresh by the time it is analysed under laboratory conditions, thereby reducing the accuracy of any results taken.

Some of the prior art analyses sweat in real time e.g. US 2017/296114. However, there is a problem of obtaining accurate data when measuring such small volumes of sweat, particularly when the flow of sweat tends to be discontinuous.

It is therefore an object of the present invention to overcome at least one of the problems in the prior art.

### SUMMARY

According to a first aspect of the present invention, there is provided a skin patch as defined in claim 1.

The skin patch of the present invention is an active measuring device that can be worn during exercise. In practice, when a bodily fluid comes into contact with the hydrophilic sensing bead, the charge concentration of the hydrophilic sensing bead changes to achieve equilibrium and is monitored by the sensor. As such, the sensor generates real-time quantitative data so as to provide real-time analysis of biometric parameters of bodily fluids such as sweat.

Furthermore, since the sensor is embedded in the hydrophilic sensing bead, the hydrophilic sensing bead can maintain electrical connectivity, even in the absence of sweat and therefore has the potential to constantly obtain data relating to the concentration of the hydrophilic sensing bead. Accordingly, fresh sweat can be monitored to obtain real-time data. This data can be utilised by comparing present data with past performances to provide a highly personalised training aid for use across a wide range of industries such as the health, sport and military industries.

According to a second aspect of the present invention, there is provided a kit as defined in claim 20.

Embodiments of the present invention will now be described by way of further example only and with reference to the accompanying drawings, in which:

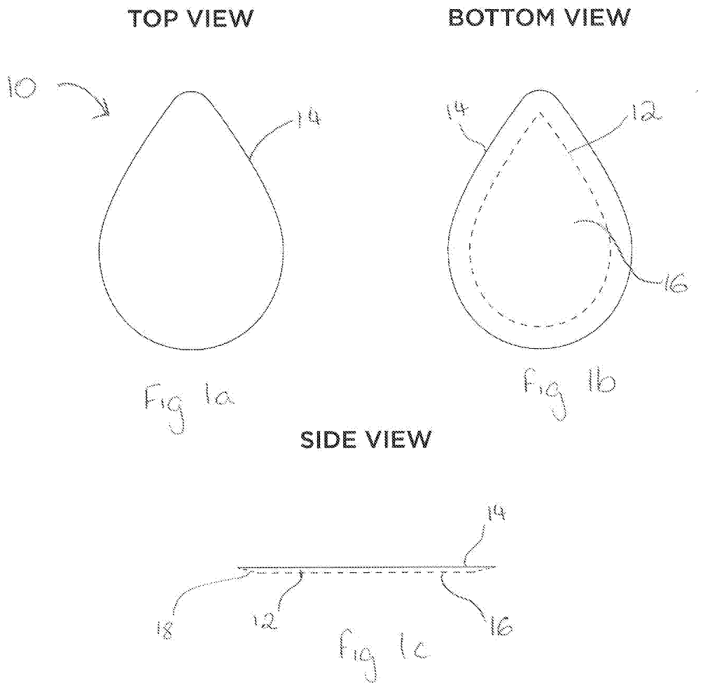

FIG. 1a is a top view of a skin patch according to a first embodiment;

FIG. 1b is a bottom view of the first embodiment;

FIG. 1c is a side view of the first embodiment;

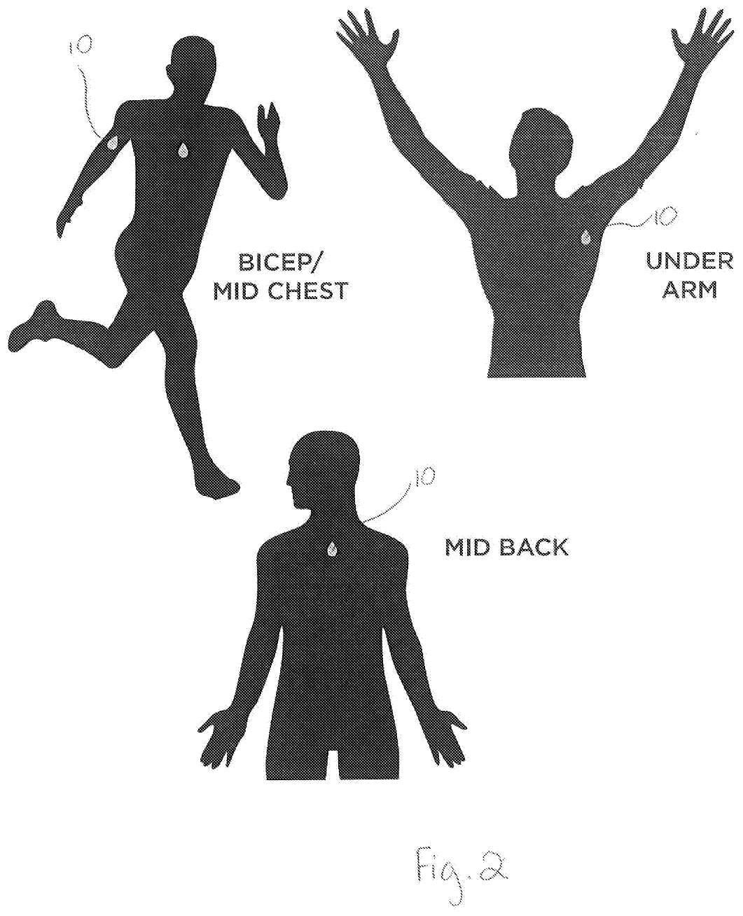

FIG. 2 is a series of schematic views of the first embodiment in use;

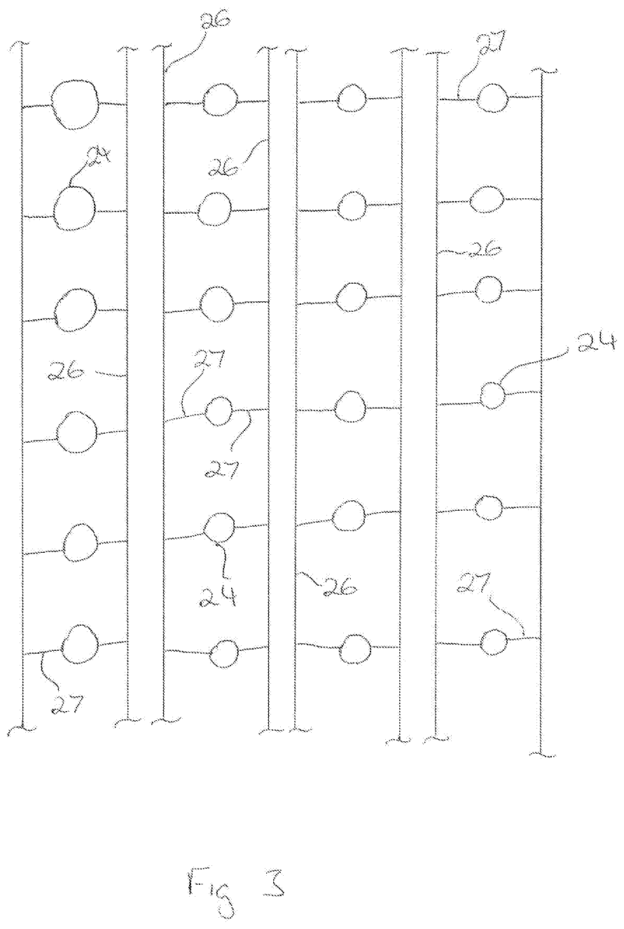

FIG. 3 is a schematic view of a sensor section of the first embodiment;

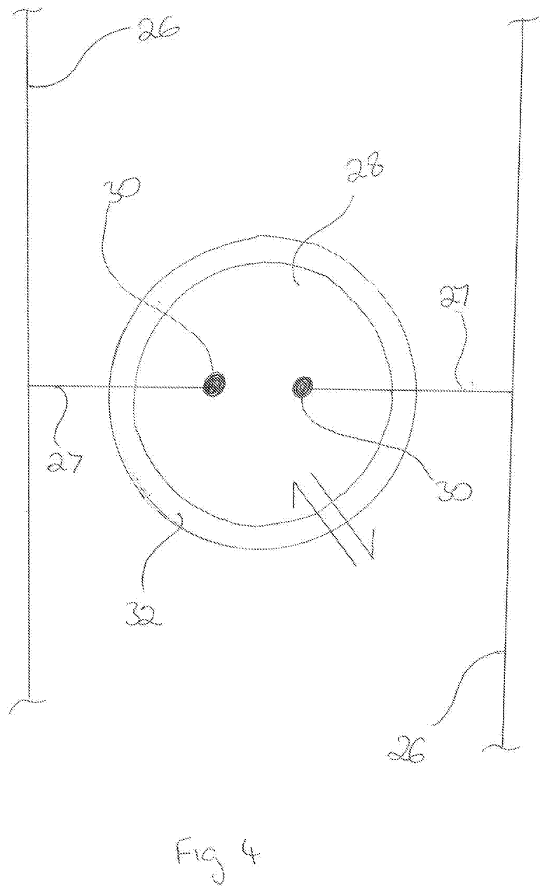

FIG. 4 is a cross-sectional view of a hydrophilic sensing bead of the first embodiment;

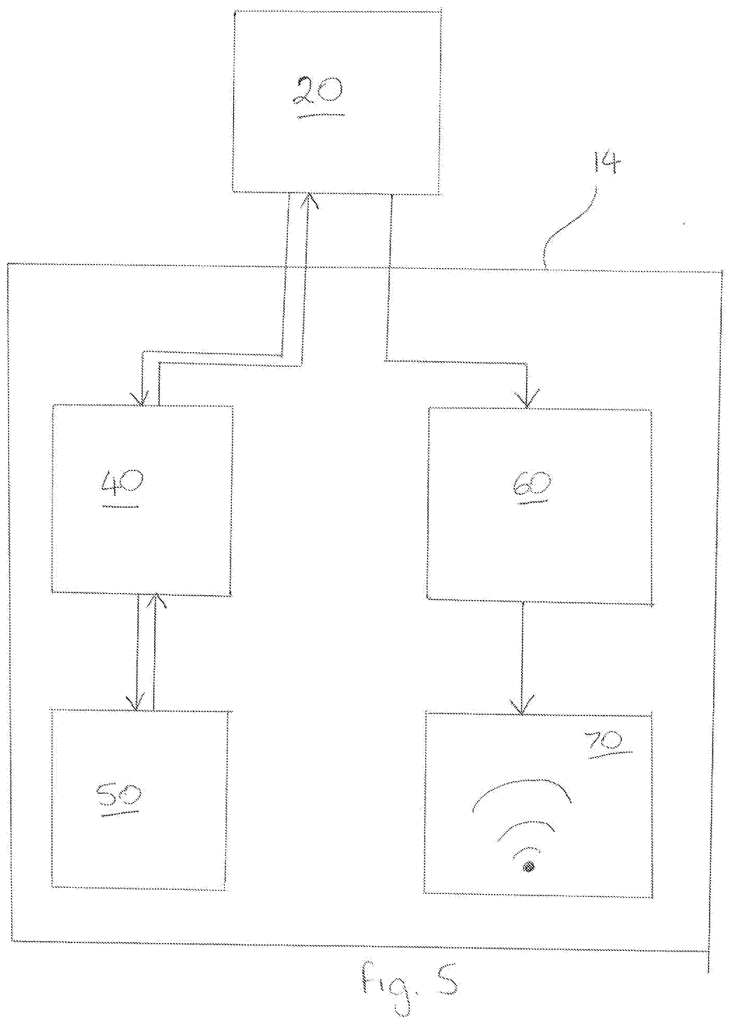

FIG. 5 is a schematic view of the second layer in communication with the sensor section of the first embodiment;

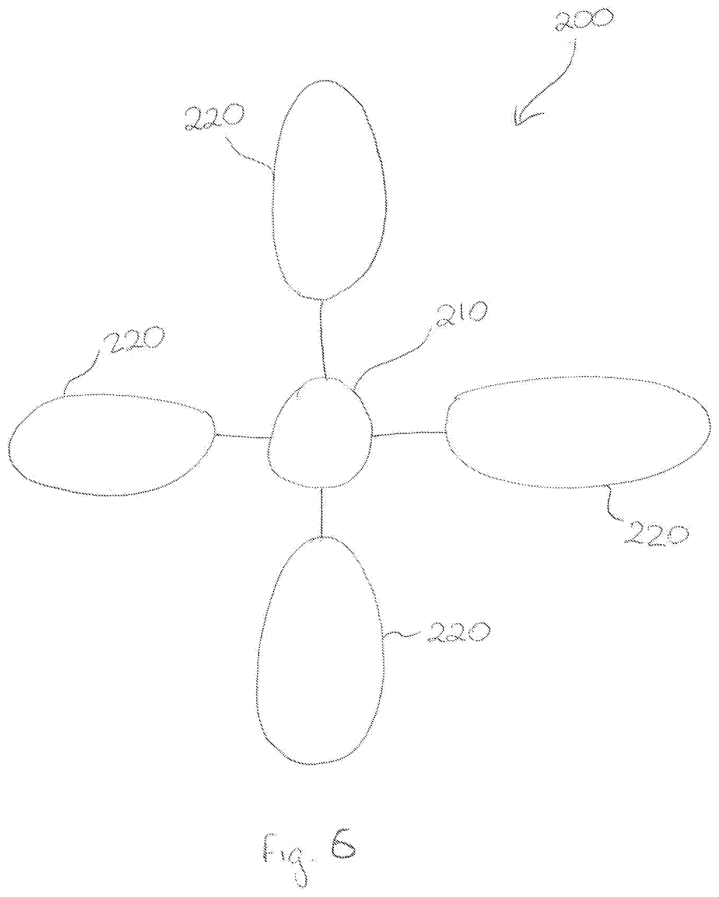

FIG. 6 is a perspective view of a sensing section of a skin patch according to a second embodiment; and

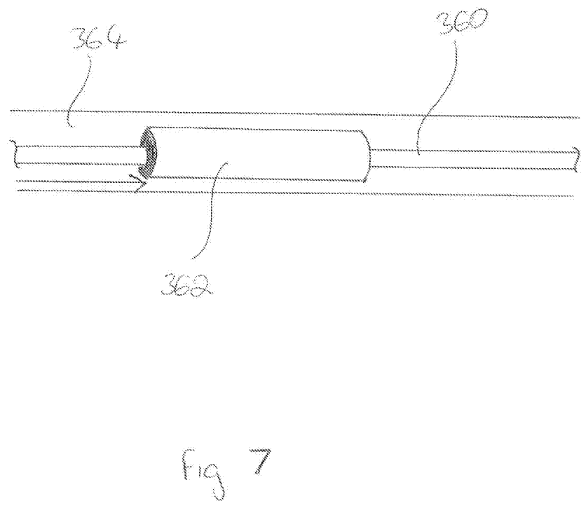

FIG. 7 is a cross-sectional view of a sensor sleeve according to a third embodiment.

### DETAILED DESCRIPTION

A first embodiment of the present invention is a skin patch that is an active measuring device for monitoring various biometric parameters such as the chemical composition of sweat. The skin patch is however not limited to such use, and may also be used to measure physiological parameters of other bodily fluids such as tears, saliva or urine.

FIGS. 1a, 1b and 1c show respective top, bottom and side views of the first embodiment of the skin patch 10, which includes a first layer 12 and a second layer 14. The first layer 12 is a lower layer that is for contacting skin in use, while the second layer 14 provides an upper layer. In use, the skin patch 10 is worn directly on a user's skin in a similar manner to a plaster or the like. The skin patch 10 has an area of 1-25 cm², preferably between 1-20 cm², more preferably between 2-15 cm² and most preferably between 2-10 cm².

As shown in FIGS. 1a and 1b, the second layer 14 is substantially planar with an overall curved ovoid pear, tear-drop or egg-shaped profile. More specifically, as shown in the top view of FIG. 1a, a lower half of the second layer 14 is rounded to provide a substantially hemispherical outline, while an upper half of the second layer 14 defines an upper rounded vertex.

As shown in FIGS. 1b and 1c, the first layer 12 is coplanar with the second layer 14 and has a first surface 16 and a second surface. The second surface of the first layer 12 contacts the second layer 14 and has substantially the same egg-shaped outline and dimensions as the second layer 14. The first surface 16 has a lower half that is substantially hemispherical and an upper half that is substantially triangular with a pointed vertex. However, it is not necessary that the first layer 12 has the same tear-drop shape as the second layer 14.

The first surface 16 is smaller in surface area than the second layer 14 and is centrally disposed over the second layer 14 when viewed from the bottom perspective as shown in FIG. 1b. The first layer 12 has a slanted portion 18 that slants from the first surface 16 toward the second layer 14. An adhesive is applied to the slanted portion 18 of the first layer 12 so that in use, when the skin patch 10 is applied to skin, the adhesive holds the skin patch 10 in place.

The skin patch may be flexible and may further be stretchable. The skin patch may include fibres of polyester, which allow the skin patch to stretch. However, the skin patch is not limited to the use of polyester fibres, and may comprise other suitable materials.

Providing the skin patch with this overall ovoid or tear-drop shape provides an ergonomic design which fits the contours of the body, thereby improving the malleability and application of the skin patch 10 to skin. Moreover, the skin patch can be applied anywhere on the body. FIG. 2 shows examples of where the skin patch 10 may be applied on the body, including under the arm and on the neck, back and chest areas.

The first layer 12 comprises a sensor section 20 having a plurality of microsensors, each of which includes at least one electrode 30. A plurality of hydrophilic sensing beads 24 is provided for the plurality of sensors, such that each sensor is embedded in each hydrophilic sensing bead 24. The sensors are configured to measure the concentration of the hydrophilic sensing beads 24.

In practice, when sweat comes into contact with a hydrophilic sensing bead 24, the sweat and the hydrophilic sensing bead 24 undergo a molecular or ionic exchange so as to reach a charge equilibrium. The sensors monitor and obtain data relating to any changes in the charge or composition of the hydrophilic sensing beads 24, and this data can be used to determine the composition of the sweat that has interacted with the hydrophilic sensing beads 24, thereby providing biometric data in real-time.

FIG. 3 shows a first embodiment of a sensor section 20 in the first layer 12 including an array of hydrophilic sensing beads 24, each of which includes a sensor. A plurality of hydrophilic sensing beads 24 of the array is connected in parallel via a pair of electrically connective strings 26 with accompanying interconnective strings 27. As shown in FIG. 3, a plurality of string pairs 26 is provided. By connecting the hydrophilic sensing beads 24 in parallel across an array of electrically connective strings 26, a larger current or voltage can be measured to improve the precision of the resulting data. Moreover, connecting each bead in parallel rather than in series ensures that if any of the hydrophilic sensing beads fail, the circuit can still operate. Alternatively, the hydrophilic sensing beads may be connected in series.

In other embodiments, however, the connectors may be provided as a grid or mesh connecting each of the hydrophilic sensing beads 24 to one another. In further embodiments, the hydrophilic sensing beads 24 are not connected to one another, so that each hydrophilic sensing bead 24 provides individual data. In yet further embodiments, the hydrophilic sensing beads are connected via flexible 2D circuits on a permeable base polymer sheet.

It is preferable for the plurality of hydrophilic sensing beads 24 to have an inter-separation distance that ensures they do not affect one another. Preferably, the inter-separation distance is equivalent to at least five times the radius of the hydrophilic sensing bead. This means that an average of the sum of the results can be obtained so as to increase the accuracy of the measurements. For example, the plurality of hydrophilic sensing beads may be distributed substantially evenly across the entire sensor section 20.

In the first embodiment, the hydrophilic sensing beads 24 are embedded within a plurality of fibres in the first layer 12. The fibres provide capillary channels that wick sweat through the skin patch 10 and past the hydrophilic sensing beads 24 i.e. towards and away from the hydrophilic sensing beads 24 in use. In this way, the fibres provide a transport section for transporting sweat or other bodily fluids to come into contact with the hydrophilic sensing beads 24 in the sensor section 20. The transport section is configured to control the rate at which a bodily fluid is transported to the sensor. The fibres also provide a removal section for expelling sweat out of the skin patch 10. In doing so, old sweat can be removed efficiently.

Preferably, the plurality of fibres includes an elastomeric material with wicking capabilities. In such embodiments, the elastomeric material may have a bilayer structure with relatively thin polyester fibres on the inside and bigger polyester fibres on the outside to create a wicking gradient from the outside to the inside. This has the advantage of making the material feel dry against skin.

The sweat patch preferably also includes a silver complex such as a silver chloride coating to stop sweat odours. Commercial examples include Polygiene®. In such embodiments, the silver chloride coating is conductive, which reduces the electrical noise felt by the hydrophilic sensing bead 24. As such, the silver chloride coating provides noise screening by improving the signal to noise ratio, and may be used to provide a reference potential. Such embodiments therefore benefit from an improved signal quality.

In some embodiments, a membrane may be formed over the device 10 of the present invention. This membrane may be formed so as to partially cover, or preferably fully cover and enclose, the patch. The membrane may be porous or non-porous to allow bodily fluids to permeate the membrane or to protect the patch from the outside environment. As such, the membrane may be permeable thus allowing passage of a bodily fluid from outside the membrane to the sensor within the patch.

In an alternative embodiment, the device 10 of the present invention need not be adhered to the user's skin or even worn directly on a user's skin and instead may be integrated into an item of clothing of the wearer/user. In such a case, the device could be woven into the material of the garment or integrally formed as part of the garment's material. When the device is integrated into an item of clothing, it may contact the user's skin or may be separated from the user's skin so that the garment wicks the bodily fluid of the user to the device 10. Alternatively, it may be placed at a position that periodically comes into contact with the user's skin so as to interact with the bodily fluid of the user. An example of this could be that the device is integrated into the arm pit portion of a user's t-shirt which is separated from the skin of the user. During physical activity the gesticulation of the user would cause the device to come into contact with the arm pit and thus interact with the bodily fluid of the user, in this case sweat. Alternatively, when the device is integrated into a user's clothing, the device may be configured to absorb any bodily fluids absorbed by the item of clothing into which the device is integrated.

FIG. 4 shows a first embodiment of a hydrophilic sensing bead 24 and a sensor embedded within the hydrophilic sensing bead 24.

As shown in FIG. 4, the hydrophilic sensing bead 24 in the first embodiment is substantially spherical. However, the hydrophilic sensing bead of the present invention is not limited to this shape, and may be formed for example as an ovoid or a hemispheroid.

The hydrophilic sensing bead 24 comprises a hydrogel 28. Each hydrophilic sensing bead 24 is small in volume and highly curved taking on an approximate spherical shape so as to minimise the signal to noise ratio of the resulting data. Thus, each bead preferably is or comprises a droplet or blob of hydrogel, which is self-contained and has a fixed volume. The radius of the hydrophilic sensing beads 24 is in the range of 1 μm-1 mm, and more preferably from 30-100 μm. The volume of the hydrophilic sensing beads 24 is in the range of 1 fl-10 μl. This increases the flux of molecular or ionic exchange at the surfaces of the hydrophilic sensing beads 24, thereby increasing the rate of charge equilibration. The flux of molecular or ionic exchange is also increased at the surface of the electrodes 30, thereby increasing the speed of response of the electrode 30 to concentration or composition changes. Accordingly, the rate of detection of any changes in charge is increased, providing a rapid response time for accurate real-time results.

The sensor is an amperometric sensor comprising two electrodes 30 embedded within the hydrophilic sensing bead 24. The amperometric sensor works by a coulometric method, in which the charge concentration of a specified variable, e.g. a biomarker molecule , in a given hydrophilic sensing bead 24 is monitored by measuring the integrated current. Specifically, the hydrogel 28 in each hydrophilic sensing bead 24 has a predetermined volume and chemical composition. Any change in the concentration of a given variable indicates any molecular or ion exchange that has taken place.

The coulometric method has the benefit of making it unnecessary to perform calibration techniques. This is because the total amount of charge obtained will be the same, irrespective of the detection efficiency of a given sensor.

The amperometric sensors can determine tissue metabolism by measuring lactate levels which are an indicator of global and local muscle fatigue and recovery. The skin patch 10 may also monitor glucose levels as a non-invasive means of measuring blood glucose.

Polymers are provided to bind the species to be measured e.g. lactate and/or glucose. In some embodiments, recognition of targeted species is performed by biorecognition moieties such as a bioprotein which has a specific binding site for the analyte. Examples include but are not limited to: enzymes, antibodies, membrane channel proteins or binding molecules, such as valinomycin (for K+ ions). In other embodiments, recognition is achieved by use of synthetic binding sites chosen to bind an analyte selectively compared to other chemical species. Examples include but are not limited to: aptamers and synthetic ionophores.

The polymers are predetermined according to the species to be measured. The polymers may be provided on the sensor electrodes.

In some embodiments, the polymer is a hydrogel, which is a highly hydrophobic polymer that incorporates a high-water content. In an amperometric sensor, the binding sites are chemically joined to this polymer to keep them within the bead. An example of a hydrogel is a hydrogel comprising 30 mg/ml albumin, 60 mg/ml PEG-DE, 2% glycerol in 0.01M PBS but other forms of hydrogel may be used and tuned to give desired properties. The hydrogel provides the continuously conductive environment that allows the beads to provide electrical conductivity at all times.

Furthermore, the amperometric sensors can be switched on and off. Switching on the electrodes 30 to make a circuit depletes the charge, and switching off the circuit allows for greater equilibration with sweat that passes the hydrophilic sensing bead 24 in cases where the rate of equilibration is slow. It is therefore preferable to periodically switch on and off the sensors, which allows smaller concentrations of measured ions in the sweat to be detected. In addition, the sensitivity of the amperometric sensor to smaller volumes of sweat can be improved. Alternatively, it is possible to provide continuous detection by keeping the electrodes 30 on.

In the first embodiment, the electrodes 30 comprise carbon, while the surfaces of the electrodes 30 are platinum-based. The electrodes 30 have a diameter of between 10 and 50 μm. Furthermore, the connectors 26 together with the accompanying interconnective strings 27 connecting each sensor are provided as wires having diameters of between 10 and 50 μm.

The invention however is not limited to amperometric sensors. In other embodiments, the sensor is a potentiometric sensor comprising a first indicator electrode embedded within the hydrophilic sensing bead 24 and a second reference electrode external to the hydrophilic sensing bead 24 that measures the difference in voltage between the first and second electrodes. The potentiometric sensor monitors the total potential change of a specified variable, e.g. potassium, sodium and/or chloride ions, in a given hydrophilic sensing bead, which indicates any ion exchange that has taken place. Such potentiometric sensors can be used to detect hydration levels.

In the first embodiment, a plurality of reference hydrophilic sensing beads is provided in the skin patch 10, which are not exposed to sweat. As such, any variation in the charge concentration is dependent on body temperature, but not on sweat. The data from the reference hydrophilic sensing beads can then be compared to hydrophilic sensing beads 24 exposed to sweat in order to perform temperature calibration. This is particularly beneficial when used during exercise as a means of compensating for any changes in skin temperature that may affect the data obtained.

The skin patch 10 of the first embodiment comprises an equilibration rate means for controlling the rate of equilibration between the hydrophilic sensing beads 24 and sweat that contacts the hydrophilic sensing beads 24. This equilibration rate means takes the form of a diffusional layer 32 or membrane that coats each hydrophilic sensing bead 24, as shown in FIG. 4. The diffusional layer may include a polymer film with a lower water content than the hydrogel. In some embodiments, the diffusional layer may be a substantially dense polymer film with nanopores distributed through the film.

In practice, the diffusional layer 32 loads molecules external to the hydrophilic sensing bead internally into the hydrogel core by a suitable mechanism. For example, in some embodiments, the diffusional layer provides a passive conduit to the internal hydrogel of the hydrophilic sensing bead.

In other embodiments, the diffusional layer 32 is selectively permeable and only allows predetermined ions or molecules to pass by osmosis. In this way, the hydrophilic sensing beads 24 can be coated with different diffusional layers 32 in order to sense different components in the sweat. This means that desired analytes may be selected to permeate into the hydrophilic sensing bead, while other molecules present in sweat that might give an interfering response can be prevented from permeating into the hydrophilic sensing bead.

FIG. 5 shows a schematic view of the second layer 14 in communication with the sensor section 20.

A control unit 40, a power source 50, a processing unit 60 and a communication unit 70 are provided in this upper second layer 14 in the first embodiment.

The control unit 40 is connected to the sensors for controlling and monitoring signals from the sensors, and the power source 50 is connected to the control unit 40 and is used for activating the sensors in the sensor section 20. In the first embodiment, the control unit 40 is a chip including an application-specific integrated circuit that can be configured to control both amperometric and potentiometric sensors. The chip has an area of between 2 and 4 mm² and more preferably 2.7 by 2.7 mm².

The processing unit 60 is connected to the electrical connectors 26 and is for receiving data from the sensors and processing the received data. In the first embodiment, the processing unit 60 includes an analogue to digital converter for digitising the received data.

In some embodiments, the control unit 40 and the processing unit 60 may be combined with one another to form a single unit.

The processed data from the processing unit 60 is input into the communication unit 70, which is wirelessly connected to a smart device, for example communicating via radio, Bluetooth, Wi-fi, etc. The communication unit 70 includes a wireless module to enable this communication, and is configured to output the data to the smart device. The smart device is typically a smart phone, a watch, a computer or a tablet having a customised application configured to compare the data with past results.

In the first embodiment, the smart device is configured to perform further processing on the data. However, in other embodiments, the processing unit 60 within the skin patch can carry out some or all processing of the data, so that the smart device need only perform partial, if any, processing.

The electronics 20, 30, 40, 50, 60, 70 in the second layer 14 may be embedded in a breathable membrane or fabric. As discussed above, the second layer 14 covers the first layer 12, so the use of such a breathable membrane or fabric allows sweat wicked by the fibres in the first layer 12 to escape from the patch 10. In addition, the breathable fabric or membrane is preferably waterproof. This keeps the patch waterproof so that it can be used in wet conditions, such as running in the rain, or even in water, for example by swimmers. A suitable breathable fabric would be, for example, made with GoreTex™, or a similar material.

Alternatively, wicking fibres in the transport section in the first layer 12 may be exposed to air through appropriately-sized apertures in the second layer 14 to allow the sweat to escape. In an another option, the wicking fibres may exposed to air at the side of the patch, below the second layer 14. This would allow the second layer 14, or a further layer (not shown) covering the second layer 14, to be waterproof but not breathable.

It is also possible to incorporate the electronics 20, 30, 40, 50, 60, 70 in a breathable, non-waterproof material, for example made of a similar material to the transport section in the first layer. This can then be covered with a third layer, which is breathable and preferably waterproof, as discussed above.

In yet another embodiment, the electronics 20, 30, 40, 50, 60, 70 may be incorporated in the first layer 12, and the second layer 14 may comprise or consist of a breathable, preferably waterproof material.

The electronics 20, 30, 40, 50, 60, 70 may also be provided in a second layer 14 of any desired material (e.g. non-breathable) but with a smaller surface area than the first layer 12. The two layers may then be covered with a third breathable, preferably waterproof fabric so that the sweat wicked through the transport layer, which holds the sensing beads 24, can escape around the second layer and through the third layer.

Some or all of the electronics 20, 30, 40, 50, 60, 70 may overlap some or all of the sensing beads 24 and/or the transport layer in plan view. In practice, it is preferred that none or only a relatively small proportion of the fibres forming the transport layer are covered with the electronics 20, 30, 40, 50, 60, 70, so that sweat can escape around the electronics 20, 30, 40, 50, 60, 70 and around/or through the second layer 14, and through the third layer, where provided.

Together with the customised application, the skin patch 10 can provide a highly personalised training and fitness aid by monitoring various biometric parameters in real-time. This is particularly advantageous for use as a personal training aid, since users such as athletes and cyclists can monitor indicators such as potassium, sodium, chloride, glucose and lactate levels as they exercise to determine the best exercise regime for their own personal health. Another use for the skin patch 10 may include monitoring team members during a game such as football to monitor the energy reserve of the whole team during a match. The skin patch 10 is not restricted to just sports and fitness training, but has wide-ranging applications across other industries, from medical to military use.

The second layer 14 may also provide the first layer 12 with physical and/or electrical protection from the external environment. This is because it is important to maintain an isolated and controlled environment for the sensor section 20 so as to improve the accuracy of the data obtained.

In the first embodiment, the skin patch 10 is disposable. However, the first layer 12 may be disposable, while the second layer 14 may be reusable with a new first layer, so as to reuse the communications unit for other patches.

The skin patch 10 can also be extended to be multi-modal, measuring temperature changes, pulse and oxygen levels for example.

The present invention is not limited to the above configurations, and can be implemented to provide various topologies.

For example, FIG. 6 shows a second embodiment of the skin patch that differs from the first embodiment in the topology of its sensor section 200 and control unit 210. The sensor section 200 comprises a plurality of sensor zones 220 surrounding a centrally disposed control unit 210.

Both the sensor zones 220 and the control unit 210 may be provided in the same layer, which makes for particularly efficient manufacture in embodiments where the skin patch is disposable. However, in other embodiments, the sensor zones 220 and the control unit 210 are disposed in different layers to one another. This is preferable if the control unit 210 is to be reused in another skin patch.

Each sensor zone 220 comprises hydrophilic sensing beads 24 embedded within fibres according to the first embodiment. Providing a plurality of sensor zones 220 in this manner is advantageous, since each sensor zone 220 can be used to detect a different variable, or as controls against each other.

In some embodiments, the elements of the first and second layers may constitute the same layer. For example, the skin patch may comprise one layer in which the electronics of the communication unit surround a central portion that includes the sensor section and transport section.

Furthermore, the skin patch may comprise a separate sweat handling layer including the transport section that contacts the skin in use. In such configurations, the first layer, which comprises the sensor section, is interposed between the sweat handling layer and the upper layer.

Additionally, the invention is not limited to the second layer including all the electronics contained within the skin patch. For example, the control unit and the power source may be provided in the first layer i.e. the same layer as the sensor section, with the processing unit and the communication unit provided in a different layer.

FIG. 7 shows a third embodiment of a sensor in the skin patch, which comprises a series of fibres, a plurality of electrically conductive wires 360 embedded within the fibres, and a sensor sleeve 362 along portions of the length of each wire 360. The sensor sleeve 362 comprises a hydrogel and a sensor in the same manner as the hydrophilic sensing beads 24 in the first and second embodiments.

The fibres define capillary channels 364 that run alongside the wires 360 so as to channel sweat past the sensor sleeves 362 as indicated by the arrow shown in FIG. 7. Accordingly, the sensor sleeves 362 that contact the channeled sweat undergo ionic or molecular exchange to equilibrate according to the same mechanism as the hydrophilic sensing beads 24 of the first and second embodiments. The sensor sleeves 362 are connected to the wire 360 that leads to a control unit, power source, processing unit and communication unit in the same manner as the first and second embodiments.

Such embodiments include potentiometric sensors. The polymer is a hydrophobic polymer such as polyvinyl chloride (PVC), which is provided as a membrane that acts as an insulating barrier to contain ionophore molecules. Such membranes are thin to make the resulting hydrophilic sensing beads efficiently responsive. Conducting polymers such as PEDOT.PSS (also known as poly(3,4-ethylenedioxythiophene) polystyrene sulfonate) may be used. Such conducting polymers have the advantage of providing a low impedance contact with high charge density to PVC membranes. Such hydrophobic polymers may be provided as polymer sleeves may also be provided on the wires. Polymer sleeves may also be provided on the wires.

The foregoing description has been given by way of example only and it will be appreciated by a person skilled in the art that modifications can be made without departing from the scope of the present invention as defined by the claims.

1. A skin patch for measuring a biometric parameter of a bodily fluid, the skin patch comprising:
a hydrophilic sensing bead and a sensor including at least one electrode embedded within the hydrophilic sensing bead, the sensor being configured to obtain data relating to the concentration of the hydrophilic sensing bead relating to the parameter;
a transport section for transporting the bodily fluid to contact the hydrophilic sensing bead; and
a communication unit including an input section communicating with the sensor via a connector, and an output section configured to output the obtained data.

2. The skin patch according to claim 1, wherein, when the bodily fluid contacts the hydrophilic sensing bead, the hydrophilic sensing bead is configured to equilibrate with the bodily fluid by molecular or ionic exchange with the bodily fluid.

3. The skin patch according to claim 1, wherein the hydrophilic sensing bead comprises a hydrogel.

4. The skin patch according to claim 1, wherein the sensor is one of a potentiometric sensor and an amperometric sensor.

5. The skin patch according to claim 4, wherein, when the sensor is an amperometric sensor, the sensor comprises two electrodes having surfaces that comprise platinum.

6. The skin patch according to claim 4, wherein, when the sensor is an amperometric sensor, the sensor comprises carbon.

7. The skin patch according to claim 1, wherein the diameter of the electrode is 10-50 μm.

8. The skin patch according to claim 1, wherein the skin patch is configured to undergo temperature calibration.

9. The skin patch according to claim 1, wherein the transport section is configured to control the rate at which a bodily fluid is transported to the sensor.

10. The skin patch according to claim 1, wherein the skin patch further comprises a removal section for expelling a bodily fluid that has passed the sensor from the skin patch.

11. The skin patch according to claim 10, wherein at least one of the transport section and the removal section comprises a plurality of capillary channels.

12. The skin patch according to claim 1, wherein the skin patch further comprises an equilibration rate means for controlling the rate of equilibration of the hydrophilic sensing bead and the bodily fluid.

13. The skin patch according to claim 1, wherein a plurality of hydrophilic sensing beads is provided.

14. The skin patch according to claim 13, wherein a connector is provided for each of the plurality of hydrophilic sensing beads.

15. The skin patch according to claim 13, wherein a connector is provided for at least two hydrophilic sensing beads, and wherein the at least two hydrophilic sensing beads are arranged in parallel along the connector.

16. The skin patch according to claim 1, wherein the output section of the communication unit is configured to output data wirelessly.

17. The skin patch according to claim 16, wherein the output section is configured to output data via one of a radio, Bluetooth and a Wi-Fi connection.

18. The skin patch according to claim 1, wherein the skin patch is curved.

19. The skin patch according to claim 1, wherein the skin patch is droplet-shaped.

20. A kit for measuring a biometric parameter of a bodily fluid, the kit comprising:
the skin patch according to claim 1; and
a device configured to communicate with the output section of the communication unit.
